(19) United States
(12) Patent Application Publication
(10) Pub. No.: US 2011/0040574 A1
(43) Pub. Date: Feb. 17, 2011

(54) Health Monitoring System with Biometric Identification

(76) Inventors: Ho Chung Nicholas Fung, Singapore (SG); Chu Yong Sang, Singapore (SG)

Correspondence Address: MCCRACKEN & FRANK LLP, 311 S. WACKER DRIVE, SUITE 2500, CHICAGO, IL, 60606, US

(21) Appl. No.: 12/918,762

(22) PCT Filed: Mar. 25, 2008
(86) PCT No.: PCT/SG08/00092

§ 371 Date: Oct. 18, 2010

(51) Int. Cl. A61B 5/024; H04L 9/32; A61B 5/00; A61B 5/08; A61B 5/145; G06Q 50/00

(52) U.S. Cl. 705/2; 713/176; 713/168; 713/175; 600/300; 600/508; 600/529; 600/365; 600/309

(57) ABSTRACT

A health monitoring method and system is disclosed. A sensor for use in a health monitoring system includes a first transducer for collecting biometric data for biometrically identifying an individual, a second transducer for measuring at least one physiological parameter and outputting data indicative thereof, and an output for outputting the data. In addition, a health monitoring system includes a server comprising a data processor and a memory, and one or more of the sensors, wherein the server is arranged to receive the data from the one or more sensors, to process the data associated with the respective individual from which the data was collected, and to output at least one result of the processing of the data.

### FIELD OF THE INVENTION

The present invention relates to a health monitoring system, of particular but by no means exclusive application as a health insurance risk-rewards management system.

### BACKGROUND OF THE INVENTION

Existing health insurance principally insures the expense associated with the treatment of the sick and unwell, and in some cases in respect of the expense of certain preventative measures. This provides, however, no incentive to the insured to preserve good health, as the cost of the insurance is generally not coupled to the health of the insured person.

At most, a reduced premium may be payable if the insured person declares that he or she is not a smoker, or otherwise undertakes to refrain from a practice known to damage health. Even then, however, health insurers must rely on the mere undertaking, or possibly on the results of a periodic but infrequent medical examination (by a suitably qualified medical practitioner who certifies the state of health of the insured), in assessing a new, reduced premium. More frequent examinations are possible but their expense and inconvenience cannot generally be justified.

Some individuals monitor their own health, such as by checking blood pressure or pulse, possibly in association with regular exercise. For example, certain existing heart beat monitors (such as those consisting of a chest strap transmitter and a wrist receiver) are adapted for private use by those with little expertise. However, records of such monitoring cannot be used by insurers as such records are not certified as authentic by a suitably qualified party (i.e. typically a medical practitioner).

### SUMMARY OF THE INVENTION

According to a first broad aspect, therefore, the present invention provides a portable sensor adapted to be secured to an individual for use in a health monitoring system, comprising:
a first transducer for collecting biometric data for biometrically identifying an individual;
a second transducer for measuring at least one physiological parameter and outputting data indicative thereof; and
an output for outputting the data with or in association with collected biometric data, so that said data can be suitably associated with said individual indicated by said collected biometric data.

The first transducer may be, for example, a fingerprint sensor, a DNA analyser, a retinal pattern sensor, an iris pattern sensor, or a face recognition sensor.

The physiological parameter may comprise, for example, rate of heart beat, rate of breathing, blood sugar content, blood alcohol content, or body fat content. Thus, the second transducer may comprise a heartbeat rate monitor, a breathing rate monitor, a blood sugar level monitor, a body fat content monitor, a blood alcohol content monitor, or a nicotine level monitor.

In principle, the first and second transducers may be provided as a single transducer, in those embodiments where the biometric data is derivable from the at least one physiological parameter. For example, some physiological parameters, even though they vary and in doing so provide an indication of the individual's exercise or well-being—are nonetheless sufficiently characteristic of the individual to be used for identification.

In one embodiment, the sensor includes an encryption module for digitally signing and/or encrypting the data before the data is outputted.

Thus, any unauthorised modification of the data may be detected once the encrypted and digitally signed data has been transmitted electronically to a validation system for verifying the authenticity of the data.

The sensor may be configured to store a registration biometric measurement for subsequent comparison with a biometric data collected by the first transducer, and arranged to collect or output the data only if the registration biometric measurement agrees with the biometric data collected by the first transducer. In other embodiments, the sensor is adapted to output the collected biometric data with or in association with the data, so that the data can be suitably associated with the individual indicated by the collected biometric data.

According to another broad aspect, the invention provides a health monitoring system, comprising:
a server comprising a data processor and a memory; and
one or more portable sensors as described above;
wherein the server is arranged to receive the data from the one or more sensors, to process the data associated with the respective individual from which the data was collected, and to output at least one result of the processing of the data.

The server may include a cryptographic unit for decrypting the data, and optionally for authenticating the data.

In some embodiments, the server includes a module for processing the data and identifying individuals with higher or lower health risk.

The server—in one embodiment—includes an incentives module with incentive data, for processing the data and determining any reward or entitlement for the individual from the data and from the incentive data.

In a particular embodiment, the server is also adapted to analyze the data, and to generate and output health advice for the individual from whom the data was collected.

Thus, the invention can be used to assist individuals by continually monitoring and record one or more physiological parameters. The data may be digitally authenticated with, for example, biometrics, so it is possible for individuals to submit authenticated health records to, for example, an insurance company for analysis and input into the identification—and possibly reward—of individuals whose lifestyles lead to lower health risks and who therefore pose a potential lower cost to the insurer. The insurer can give rewards to such individuals as incentives for them to maintain healthy lifestyles. Such a program can be structured to reward individuals for exercising regularly and keeping to a healthy diet, thereby reducing the costs of health insurance to both individuals and insurers.

Such a system could also be used by employers who would be interested in promoting healthy lifestyles amongst their staff for better productivity. Military agencies that need to monitor the health of their servicemen could use the system of the invention to allow servicemen to record their own exercise, and to submit records of that exercise electronically to the agencies without having to undergo physical examination by a medical examiner. Fitness clubs may also use such a system to promote and track the exercise of club members without direct physical intervention, and give club members incentives and rewards in return.

In another broad aspect, the present invention provides a method of monitoring health employing the portable sensor or system described above.

In a further, broader aspect of the present invention, the health monitoring system (“the System”) is operated by a trusted third party (“Trusted Service Provider”) who provides monitoring services to a plurality of parties such as insurers and employers (“Service Consumers”). In such an embodiment, the Trusted Service Provider operates the System and issues bio-sensors to registered individuals for collection of health metrics. Service Consumers subscribe to monitoring service and are therefore relieved of the burden of having to manage and operate the System. Such an embodiment further benefits individuals who then need only register with a single monitoring service to meet the needs of a plurality of Service Consumers. An individual specifically authorises said Trusted Service Provider to release individual's said health metrics to selected Service Consumers, depending on individual's agreement with the Service Consumer. Such authorisation may be in the form a physical letter or consent agreement, or in digital form, where individual issues to Service Consumer a digital certificate, preferably through said bio-sensor, containing the appropriate authorisation information authorising Trusted Service Provider to release selected parts of said health metrics to the Service Consumer upon the Service Consumer submitting said digital certificate to Trusted Service Provider. Said digital certificate may contain information such as expiry dates beyond which said digital certificate would be invalid and specifications of the parts of said health metrics to be released. The Trusted Service Provider uses said System to validate said digital certificate submitted by Service Consumers and said System is configured to release parts of said health metrics information depending on the validity and specification contained in said digital certificate.

### BRIEF DESCRIPTION OF THE DRAWING

In order that the invention may be more clearly ascertained, embodiments will now be described, by way of example, with reference to the accompanying drawing, in which:

FIG. 1 is a schematic view of a health monitoring system according to an embodiment of the present invention;

FIG. 2 is schematic view of a bio-sensor of the system of FIG. 1 according to the present invention;

FIG. 3 is schematic view of the server of the system of FIG. 1 according to the present invention; and

FIG. 4 is a flow diagram of the method implemented by the system of FIG. 1 according to the present invention.

FIG. 5 illustrates an embodiment involving a Trusted Service Provider providing health monitoring services to a plurality of Service Consumers and individuals.

### DETAILED DESCRIPTION OF THE INVENTION

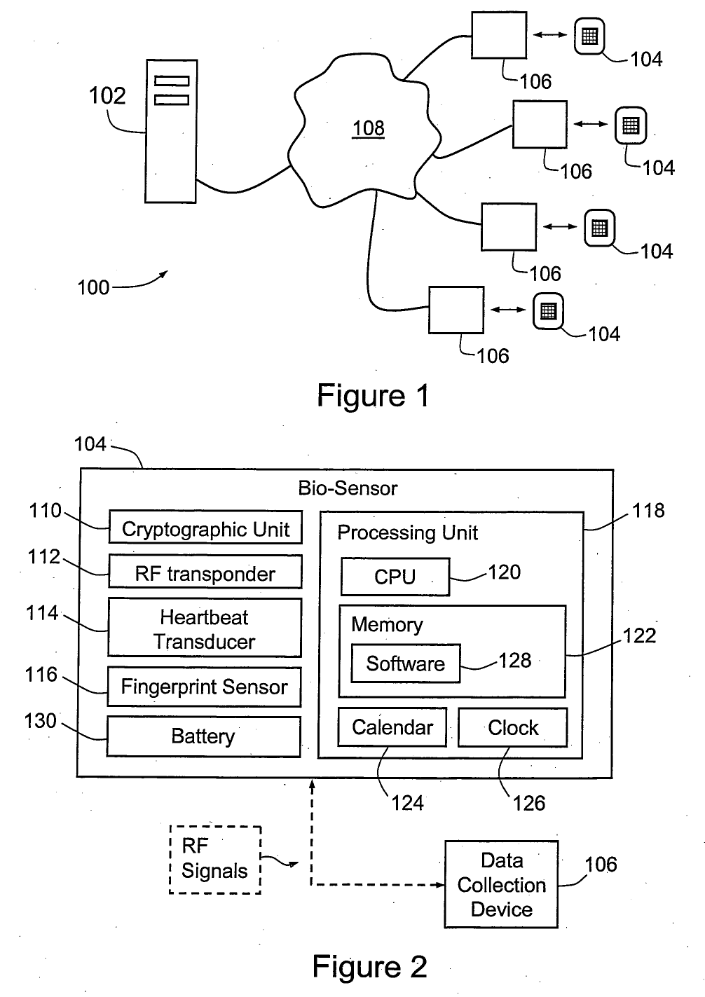

FIG. 1 is a schematic view of a health monitoring system 100 according to an embodiment of the present invention. System 100 includes a server 102 and a plurality of bio-sensors 104 and data collection devices 106. Data collection devices 106 are in data communication with server 102 via, for example, the internet 108. Each bio-sensor 104, as is explained in greater detail below, communicates with a corresponding data collection device 106 by RF signals. By this mechanism, data collected by bio-sensors 104 is uploaded to data collection devices 106 and from there to server 102.

Bio-sensors 104 are adapted to be worn by individuals who have health insurance (with an insurer); the insurer provides system 100 to facilitate the collection of information germane to the health insurance of its insured clients. It will be appreciated by those in the art, however, that system 100 could be used to collect health data for other purposes, such as to assist a government health department to survey a population.

FIG. 2 is a schematic view of a bio-sensor 104. Each bio-sensor 104 comprises a cryptographic unit 110 containing cryptographic keys generated and encoded into the cryptographic unit 110 by the issuer (typically the insurer) of the bio-sensor 104 and processing hardware and logic for digitally signing data recorded by bio-sensor 104, a radio frequency (RF) transponder 112 (for communicating and exchanging data with a corresponding data collection device 106 by RF signal), a transducer 114 for measuring heartbeat rate and a fingerprint sensor 116.

Each bio-sensor 104 also includes a processing unit 118 that includes a CPU 120, memory 122, a clock 124 and a calendar 126. Memory 122 includes software 128 executable by CPU 120 for controlling the respective bio-sensor 104. Each bio-sensor 104 is powered by an electrical power source in the form of a battery 130, though it will be appreciated that other power sources may be used in other embodiments (such as photovoltaic cells or, where the bio-sensor need not be portable, mains power).

Software 128 controls bio-sensor 104 to initially detect and register one or more fingerprints of the insured person that has been assigned, or otherwise provided with, that specific bio-sensor 104. Bio-sensor 104 will perform further health data measurements only when it subsequently detects that it is mounted on a finger of the insured person, by sensing the fingerprint thereof with fingerprint sensor 116. It will be appreciated by those skilled in the art that the initially registered fingerprint data may be stored in a parameterized form, and compared with the similarly parameterized fingerprint data at the start of each subsequent metrics data collection. If this comparison fails (possibly after a number of repetitions of fingerprint measurement and comparison), bio-sensor 104 suspends operation, at least for a predefined period.

In use, therefore, bio-sensor 104 records metrics data of the person whose fingerprint or prints have previously been registered with and recorded on bio-sensor 104. This metrics data is subsequently uploaded to the corresponding data collection device 106, and transmitted by that corresponding data collection device 106 to server 102 over, in this embodiment, the internet 108. The insurer can then validate the metrics data and determine from that data the extent to which the insured person has met the conditions necessary for the insured person to be entitled to incentive rewards.

In this embodiment, the metrics data comprises the average heartbeat rate over hourly periods; these data are measured and recorded by bio-sensor 104. These data are transmitted to the corresponding data collection device 106 upon receiving from the collection device 106 an RF signal adapted to prompt bio-sensor 104 to do so. The insured individual is thus monitored, which enables the insurance company to determine whether the individual is indeed following an appropriate exercise regime and therefore entitled to incentives.

Each bio-sensor 104 may optionally be provided with other sensors, such as a sensor for determining blood sugar level and other blood-related metrics that can be used to track an individual's dietary habits and hence the corresponding entitlement to benefits. Other embodiments substitute for fingerprint sensor 116 sensors that detect other types of biometrics to identify an individual person, such as a miniature camera mounted on spectacles to scan the wearer's iris or retina.

In still another embodiment, transducer 114 is instead adapted to measure other health indicators of the individual, such as the breath alcohol level, rate of breathing or body fat content. Such measures, referred to hereinafter as metrics, can be used to detect how much an insured exercises or drinks alcohol, so as to provide an indication of how healthy the insured person's lifestyle is. The insurer can thereby set targets that the insured person's metrics must satisfy in order to be rewarded with incentives.

In another embodiment, each bio-sensor 104 is fabricated in the form of a pill coated with an inert material and injected into the individual's body. Such bio-sensors 104 authenticate the identity of the insured persons by automatically analysing the DNA of the body tissue they are embedded in, and also include transducers that capture metrics by sampling blood. The metrics can include blood sugar levels, cholesterol levels, and other health measures that can aid the insurer to provide to the insured person better and more appropriate incentives to maintain a healthy lifestyle and hence reduce health maintenance costs.

Each bio-sensor 104 is programmed to continuously monitor and record the metrics for which—in the various embodiments—it is adapted to determine, and output the data in encrypted and digitally signed metric data records to the data collection device 106 when the data collection device 106 polls its corresponding bio-sensor 104 with RF signals. The data records are encrypted and digitally signed using cryptographic keys issued by the insurer and previously loaded into the bio-sensor's cryptographic unit 110 by the insurer.

Bio-sensor 104 is configured to package the metrics data that it measures into metric data records; it is these records that are uploaded and ultimately transmitted to server 102. A single metric data record may include more than one set of metrics data, but in this embodiment the data of each data measurement is stored in a separate metric data record. Table 1 summarises the contents of such a metric data record uploaded by a bio-sensor 104 to its corresponding data collection device 106, and transmitted to server 102.

TABLE 1
Metric Data Record

| Data Description | Remarks |
|---|---|
| Metrics Data | Data measured by bio-sensor, in this embodiment |
| | comprising average heartbeat |
| Insured Policy Number | Encoded into the bio-sensor by insurer prior to bio-sensor |
| and/or other unique | being issued to insured person: identifies the insured |
| identification code of | person to the insurer |
| the individual | |
| Date and time of Metric | Data is generated by the bio-sensor at the time this set of |
| Capture | Metrics is captured |
| Metric Type | Code that indicates the type of Metric that is being |
| | recorded in this data record. For example, Metric Type A |
| | indicates that this record indicates the number of |
| | continuous minutes during which the heart beat is more |
| | than 100 times in a minute. Metric Type B indicates that |
| | this record indicates the continuous minutes in a day |
| | during which the wearer's breathing rate was more than X |
| | per minute where X is a threshold value set by the Insurer. |
| Signature | A digital signature generated by the Cryptographic Unit in |
| | the bio-sensor using the rest of the data in this record and |
| | the cryptographic key provided by the Insurer in the |
| | Cryptographic Unit. This Signature authenticates this data |
| | record and assures the Insurer that the data contained in |
| | this record is genuine. |

The bio-sensors 104 are adapted to store a plurality of such metric data records. As described above, the records are uploaded from the bio-sensors 104 to the corresponding data collection devices 106, which use RF signals to induce the bio-sensor 104 to transmit the metric data records.

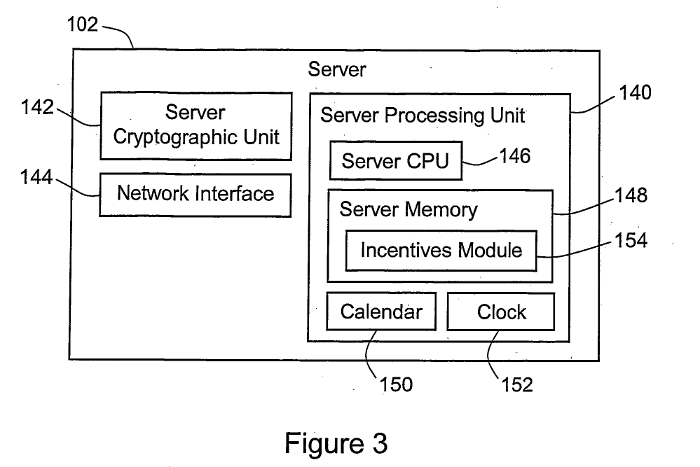

The data thus collected is sent by the data collection devices 106 to the insurer automatically, by uploading the data to server 102 over a secure internet connection. Server 102 is depicted schematically in FIG. 3. Server 102 includes, inter alia, a server processing unit 140, a server cryptographic unit 142 and a network interface 144. Server cryptographic unit 142 performs corresponding functions to cryptographic unit 110 of bio-sensor 104: when encrypted data is received from a bio-sensor 104, server cryptographic unit 142 verifies the authenticity of the data by decrypting the data and checking the digital signature with the counter-parts of the cryptographic keys that the insurer previously loaded in bio-sensor 104.

Server processing unit 140 includes a server CPU 146, server memory 148, a calendar 150 and a clock 152. The key counter-parts are stored in server memory 148, which is also provided with an incentives module 154. Incentives module 154 includes both incentives data and incentives software, which together may be described as constituting the insurer's incentive program; the incentives software is adapted to be executed by server CPU 146 and thereby to determine each insured person's rewards or other entitlements based on the incentives data and the metrics data (received from a bio-sensor 104).

The insurer publishes a schedule of rewards versus bio-sensor readings, so that insured persons can ascertain how they may design their health regime to optimize their reward or entitlements. This publication forms a part of insurer's marketing, and also forms part of the ultimate contract between insurer and insured person. Bio-sensor readings are calibrated according to the insurer's assessment of the health risks (or reduction in risks) arising from the readings. For example, readings consistent with physical exercise (say, at least 60 minutes of daily activities when heartbeat and breathing rate exceed 30% of the resting rates) for a period of 12 months entitle the insured person to a 5% discount on the annual premium. Readings showing preferred blood sugar levels over the same period will further entitle the insured person to a discount of 2%. If all readings meet the desired levels, an additional bonus discount of 3% is given. Alternatively, or additionally, the rewards may be in the form of subsidised membership to gyms or health clubs, health food supplements, etc. This scheme is automated, as the metric data records are transmitted electronically to server 102 for processing and determination of rewards. The premium discount, for example, is automatically calculated and the insured person is billed the premium reduced by that discount, if any. The insured person may also be given the choice of visiting the partner merchant facility to claim the reward (e.g. a gym membership), where the partner merchant is provided with online access to server 102 to determine the insured person's entitlements and to process the claim. In another embodiment, system 100 includes a plurality of smart cards (or chip cards) each of which is provided to a respective insured person. Each smart card, when issued to the respective insured person, includes a digital record of any rewards (e.g. a gym membership) that have been awarded to the card's owner, thereby allowing the insured person to claim the reward at a partner merchant offline.

When presented with the card by the insured person, the merchant employs a card reader to read the reward record from the smart card, thereby ascertaining the insured person's entitlement. Optionally, server 102 and the card reader may communicate electronically, whether in real-time or through subsequent synchronization, so that server 102 is apprised of reward redemptions.

The insurer's incentive program, according to this embodiment, is summarized in Table 2.

TABLE 2
Incentive Programs

| Metric and target | Reward |
|---|---|
| For age group X and gender Y, at least B minutes every | N₁ % discount on |
| 24 hours during which heartbeat rate is more than C beats | annual premium |
| per minute followed by a heart recovery rate of D beats | |
| per minute over a 12 month period. | |
| For age group X and sex Y, at least E minutes every 24 | N₂ % discount on |
| hours during which heartbeat rate is more than F beats per | annual premium & one |
| minute followed by a heart recovery rate of G beats per | free medical check-up |
| minute over a 12 month period. | |
| For age group X and gender Y, body fat content is not | M reward points |
| more than Z % at every weekly sample over a 12 month | redeemable for a gift |
| period. | or insurance premium |
| For age group X and gender Y, breath alcohol content | Free gym membership |
| does not exceed Z % at any time during the year. | for B months at C's |
| | Gym |

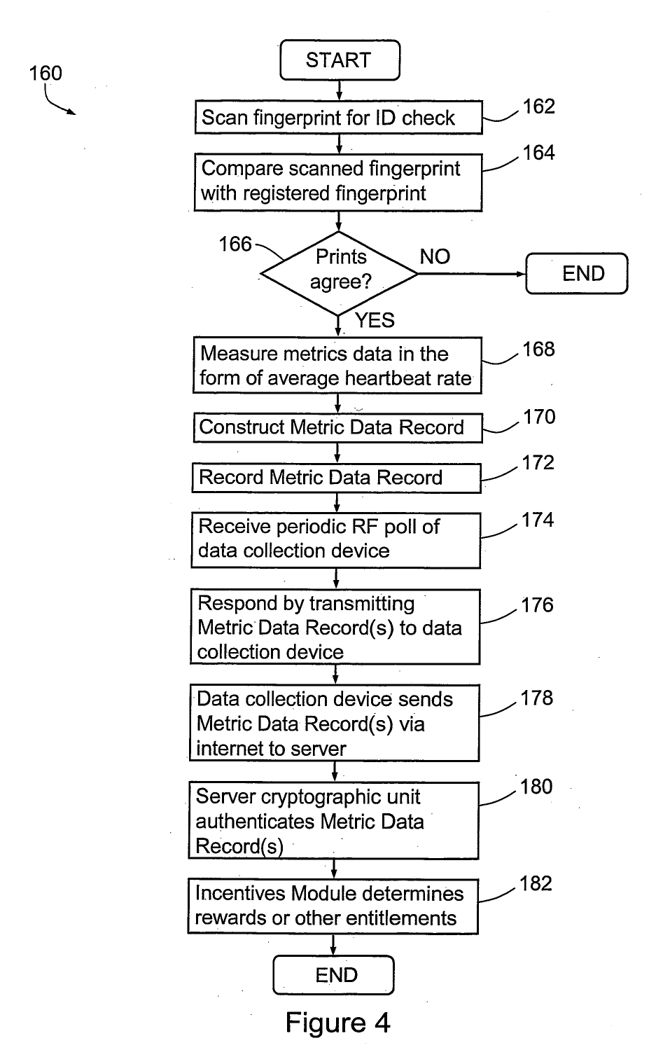

The method of this embodiment, as implemented by system 100, is summarized in flow diagram 160 in FIG. 4. At step 162, a bio-sensor 104—worn by an insured person—performs a fingerprint scan with fingerprint sensor 116 as an identity check. At step 164, bio-sensor 104 compares the scanned fingerprint with a previously registered fingerprint of the insured person, to check whether the insured person is indeed the current wearer of the bio-sensor. At step 166, bio-sensor 104 determines whether these fingerprints prints agree; if not, processing ends. If they do agree, processing continues at step 168, where bio-sensor 104 measures metrics data in the form of average heartbeat rate over a period of an hour and, at step 170, constructs a metric data record comprising the measured metrics data and various other data as described above. At step 172, bio-sensor 104 records the metric data record to memory 122.

Steps 162 to 172 may be repeated plural times but, at some point after one or more repetitions, bio-sensor 104 receives—as shown at step 174—the periodic RF polling of data collection device 106. At step 176, bio-sensor 104 responds to the polling by transmitting any metric data records not yet uploaded to corresponding data collection device 106. Upon receipt, at step 178 corresponding data collection device 106 transmits the metric data record or records via a secure internet connection to server 102. At step 180, server cryptographic unit 142 authenticates (i.e. checks the authenticity of) the metric data record or records and, if found authentic, at step 182 incentives module 154 determines rewards or other entitlements due to the insured person, based on the incentives data and the contents of the metric data record or records.

Server 102 is also adapted to analyze the metrics data that it receives, and to provide the results of that analysis—in the form of personalised advice—to the insured individual, in order to provide some guidance to the individual to observe a healthier lifestyle. This advice is transmitted by server 102, via the internet 108, to the appropriate data collection device 106. The advice is prepared based on parameters loaded into sever 102 that are accepted as valid guidelines for healthy diet or activity.

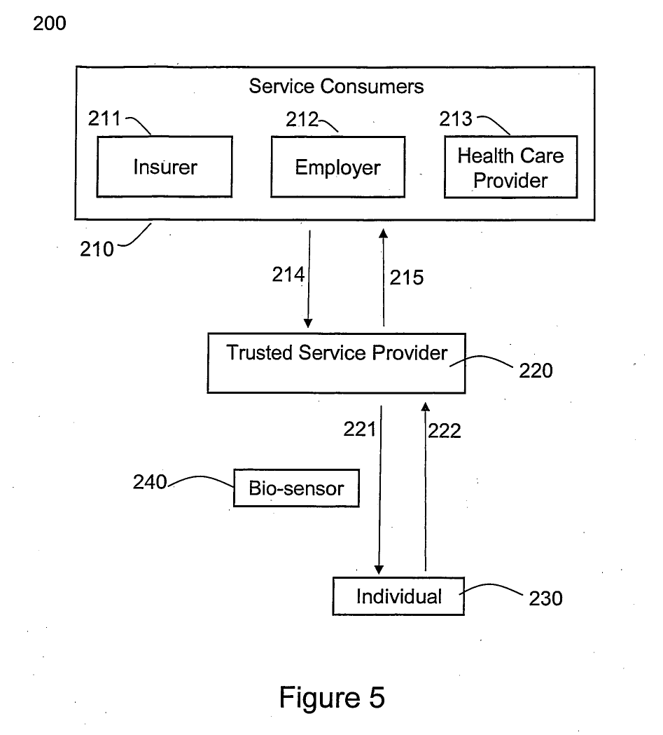

FIG. 5 illustrates an embodiment of a complete service arrangement (200) comprising a Trusted Service Provider (220) providing monitoring services to a plurality of Service Consumers (210) comprising parties such as Insurers (211), Employers (212) and Health Care Providers (213), and to a plurality of registered individuals (230) who wish their health and proof of healthy habits to be monitored. A contract (214) is signed between Service Consumer and Trusted Service Provider for said Trusted Service Provider to release metrics information (215) to said Service Consumer of said registered individuals (230) who authorise said Trusted Service Provider to do so. Said Trusted Service Provider (220) issues (221) bio-sensors (240) to individuals (230) who register (222) with said Trusted Service Provider (220). Said metrics information collected by said bio-sensors (240) from said registered individuals (230) are sent to said Trusted Service Provider, who then releases the metrics information (215) to said Service Consumer (210) authorised by said individuals (230) to receive said metrics information (215)

Modifications within the scope of the invention may be readily effected by those skilled in the art. It is to be understood, therefore, that this invention is not limited to the particular embodiments described by way of example hereinabove.

In the claims that follow and in the preceding description of the invention, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments of the invention. Further, any reference herein to prior art is not intended to imply that such prior art forms or formed a part of the common general knowledge.

1. A portable sensor adapted to be secured to an individual for use in a health monitoring system, comprising:
a first transducer for collecting biometric data for biometrically identifying an individual;
a second transducer for measuring at least one physiological parameter and outputting data indicative thereof; and
an output for outputting said data with or in association with collected biometric data, so that said data can be suitably associated with said individual indicated by said collected biometric data.

2. A portable sensor as claimed in claim 1, wherein said first transducer is a fingerprint sensor, a DNA analyser, a retinal pattern sensor, an iris pattern sensor, a face recognition sensor.

3. A portable sensor as claimed in claim 1, wherein said second transducer is a heartbeat rate monitor, a breathing rate monitor, a blood sugar level monitor, a body fat content monitor, a blood alcohol content monitor, or a nicotine level monitor.

4. A portable sensor as claimed in claim 1, wherein said sensor includes an encryption module for digitally signing and/or encrypting said data before said data is outputted.

5. A portable sensor as claimed in claim 1, wherein sensor is configured to store a registration biometric measurement for subsequent comparison with a biometric data collected by said first transducer, and arranged to collect or output said data only if said registration biometric measurement agrees with said collected biometric data.

6. A portable sensor as claimed in claim 1, wherein said sensor is adapted to be worn by the individual.

7. A health monitoring system, comprising:
a server comprising a data processor and a memory; and
one or more portable sensors as claimed in claim 1;
wherein said server is arranged to receive said data from said one or more sensors, to process said data associated with said respective individual from which said data was collected, and to output at least one result of said processing of said data.

8. A system as claimed in claim 7, wherein said server includes a cryptographic unit for decrypting said data, for authenticating said data, or for both decrypting and authenticating said data.

9. A system as claimed in claim 7, wherein said server includes a processing module for processing said data and identifying individuals with higher or lower health risk.

10. A system as claimed in claim 7, wherein said server includes an incentives module with incentive data, for processing said data and determining any reward or entitlement for said individual from said data and from said incentive data.

11. A system as claimed in claim 7, wherein said server is adapted to analyze said data, and to generate and output health advice for the individual from whom the data was collected.

12. A system as claimed in claim 1, wherein a trusted service provider operates said health monitoring system, issues said sensors to registered individuals, and collect and validate data collected from said sensors, and releases said data to selected service consumers authorised by said individuals to receive said data of said individual.

13. A system as claimed in claim 12, wherein said individual uses said sensor to issue digital certificates containing authorisation information authorising selected said service consumers to receive said data from said trusted service provider, and wherein said trusted third party validates said digital certificate and releases said data according to the authorisation contained in said digital certificate.

14. A system as claimed in claim 12, wherein said authorisation is given by individual to said service consumer and/or said trusted service provider in physical paper form.

15. A method of monitoring health, comprising:
providing an individual with a portable sensor adapted to be secured to the individual that comprises a first transducer for collecting biometric data for biometrically identifying an individual, a second transducer for measuring at least one physiological parameter and outputting data indicative thereof, and an output for outputting said data with or in association with collected biometric data, so that said data can be suitably associated with said individual indicated by said collected biometric data;
receiving said data from said sensor at a server;
processing said data associated with said individual with the server; and
outputting from said server at least one result of said processing of said data.
